(19) United States
(12) Patent Application Publication
(10) Pub. No.: US 2013/0345682 A1
(43) Pub. Date: Dec. 26, 2013

(54) Corneal Contact System

(71) Applicants: Markus Hailmann, Sachsenhausen (DE); Florian Dambacher, Haag (DE)

(72) Inventors: Markus Hailmann, Sachsenhausen (DE); Florian Dambacher, Haag (DE)

(21) Appl. No.: 13/529,680

(22) Filed: Jun. 21, 2012

(51) Int. Cl. A61B 19/00; A61F 9/008

(52) U.S. Cl. CPC A61B 19/56; A61F 9/008
USPC 606/4; 29/428

(57) ABSTRACT

An interface device for selectively engaging a suction ring with a patient interface of a laser unit includes an annular shaped base member that is formed with an orifice. The suction ring is affixed against one side of the base member to surround the orifice. A grip is mounted on the other side of the base member. Structurally, the grip includes independently manipulated handles that can be operated to symmetrically apply equal and opposite forces against the patient interface of the laser unit during an engagement of the device with the patient interface. With this engagement, the base member and the suction ring are held on the patient interface of the laser unit.

### FIELD OF THE INVENTION

The present invention pertains generally to interface devices that selectively engage a suction ring with a patient interface of a laser unit for use in stabilizing an eye of a patient relative to the laser unit during ophthalmic surgery. More particularly, the present invention pertains to interface devices which provide for a symmetric closing of the device onto a patient interface of a laser unit. The present invention is particularly, but not exclusively, useful as an interface device which avoids, or at least minimizes, the application of uneven force loadings on the optics of a patient interface of a laser unit that might otherwise compromise the operational accuracy and precision of the laser unit after the interface device has been engaged with the patient interface of a laser unit.

### BACKGROUND OF THE INVENTION

In an ophthalmic surgical procedure, wherein a laser unit is used to alter tissue of an eye, it is essential that the eye be properly stabilized in its alignment with the laser unit. In many instances, such stabilization may be best accomplished by placing the eye in direct contact with a patient interface of the laser unit. Typically, this requires a stabilizing device, such as a suction ring, that can be positioned directly against the eye (e.g. against the cornea). An interface device is then engaged with the stabilizing device and is used to fixedly hold the stabilizing device (e.g. the suction ring) relative to the laser unit. For example, U.S. Pat. No. 7,955,324 which issued to Melcher et al. for an invention entitled “Cornea Contact System,” and which is assigned to, the same assignee as the present invention, discloses a system for this purpose.

As implied above, the function of an interface device in a surgical laser procedure is essentially two-fold. For one, the interface device is used to stabilize the eye relative to the laser unit. For another, the interface device is used to align the eye with the laser unit. In order to effectively achieve both of these objectives, it is first necessary to maintain the desired optical alignment of the laser unit during the engagement of the eye with the laser unit. Preferably this can be done by avoiding the application of uneven or unopposed forces that may be inadvertently or unintentionally applied against the laser unit during the engagement. It is also desirable during such an engagement that torsional forces against the laser unit be avoided. Further, after a surgical procedure has been completed, it is desirable that the laser unit be disengaged quickly and easily from the eye, without creating unwanted forces on either the eye or the laser unit.

In light of the above, it is an object of the present invention to provide an interface device for engaging a suction ring with a patient interface of a laser unit that symmetrically applies equal and opposite forces against the laser unit during the engagement of an eye with the laser unit. Another object of the present invention is to provide an interface device for engaging a suction ring with a patient interface of a laser unit that avoids the application of torsional forces against the patient interface during an engagement of the suction ring with the patient interface of the laser unit. Still another object of the present invention is to provide an interface device that can be quickly and easily activated for disengagement of the device from a patient interface of a laser unit. Yet another object of the present invention, is to provide an interface device for engaging a suction ring with a laser unit that is easy to use, is relatively simple to manufacture, and is comparatively cost effective.

### SUMMARY OF THE INVENTION

In accordance with the present invention, an interface device is provided for selectively engaging a suction ring with a patient interface of a laser unit. Structurally, the interface device includes an annular shaped base member that defines a plane and is formed with an orifice. The base member has a first side and a second side, with the suction ring affixed against the second side of the base member to surround the orifice, for example, with an interference fit. Alternatively the suction ring may be glued to the base member. Further, as an additional possibility, the base member can be injection molded using a relatively hard material, and the suction ring can be directly injection molded onto the base member, wherein the suction ring is a relatively soft, material.

A grip that includes two independently manipulated handles is mounted on the first side of the base member. Two pivot posts are also included as part of the grip. In detail, the two pivot posts are mounted perpendicular to the plane of the base member, and are parallel to each other in a side-by-side relationship on the first side of the base member. Each of the handles is respectively engaged with one of the pivot posts for rotation on the base member. Further, each of the handles is formed with an arcuate gripping surface that is positioned so that when the handles are rotated toward each other, the gripping surfaces will independently, but cooperatively, engage the patient interface of the laser unit. It is an important aspect of the present invention that neither of the handles will apply an effective force on the patient interface of the laser unit until they are both in engagement with the patient interface of the laser unit. Once they are both in engagement with the patient interface of the laser unit, however, the handles will symmetrically apply equal and opposite forces against the patient interface of the laser unit. As intended for the present invention, this cooperation of structure is provided to avoid the application of uneven or unequal forces against the patient interface of the laser unit during an engagement of the suction ring with the patient interface.

In addition to the grip, the interface device of the present invention also includes a latching mechanism. In detail, this latching mechanism has two latch members that are separately mounted on a respective handle. Operationally, these latch members engage each other to hold the interface device in a “closed” (first) configuration. When the device is in its “closed” configuration, the respective arcuate gripping surfaces of the handles are in engagement with the patient interface of the laser unit to firmly hold the interface device on the patient interface of the laser unit.

Along with the latching mechanism, the interface device also includes a release mechanism that is operationally interrelated with the latching mechanism. More specifically, both mechanisms include a cantilever that extends from the end of one handle. This cantilever has a tab that is formed at the end of the cantilever, with one of the latch members being mounted on the tab. The cantilever itself is biased to engage the latch member on the tab with the latch member that is on the other handle. This engagement of the latching mechanism then holds the interface device in its first configuration. When the latch members are engaged with each other, however, the tab can be pulled to disengage one latch member from the other. With this disengagement the device moves into an “open” (second) configuration. In this “open” configuration the respective arcuate gripping surfaces of the first and second handles are distanced from the patient interface of the laser unit to release the interface device from the patient interface of the laser unit.

As an additional structural feature of the present invention, the interface device includes a pair of wing arms that is mounted on the base member. Specifically, these wing arms are located diametrically across the orifice from the pivot posts, and they respectively extend away from each other in opposite directions tangential to the orifice. Each handle receives one of the wing arms in a slot that is located on the handle adjacent its arcuate gripping surface. For this combination, each wing arm is formed with a protrusion, and the slot in each handle is formed with a hole for receiving the respective protrusion. The interaction of a protrusion on a wing arm, with a hole in the slot on a handle, is specifically established to limit the extent to which the handles can separate from each other when the device is in its “open” (second) configuration.

Another structural feature of the present invention involves a pair of extension springs that interact to bias the interface device toward its “open” (second) configuration. Each extension spring is mounted on a respective handle and they are oriented for contact with each other when the interface device is closed into its first configuration. Specifically, this contact generates a spring force that acts in opposition to the latch means, when the device is in the first configuration. Consequently, this spring force provides for a movement of the device into the open, second configuration when the tab on the cantilever is pulled to release the interface device from its first configuration.

Yet another structural feature of the present invention is provided to prevent, or minimize, any torsional forces that might otherwise be generated by the interface device against the laser unit. This feature involves two flange units, each of which are mounted on a respective handle and oriented to establish an overlapping engagement with each other. Structurally, each flange unit has a long flange and a short flange. Within their unit, the flanges are mounted adjacent and substantially parallel to each other. Further, the flanges are arranged so that when the units are in their overlapping engagement the long flange of each flange unit overlaps the short flange of the other flange unit with the mutually overlapping engagement of the flange units, torsional movements between the handles are prevented when the interface device is in the “closed” first configuration.

In one embodiment of the present invention, an arrangement is provided for pre-centering the patient interface with the orifice of the base member before the handles are moved to place the grip in its “closed” configuration. With this pre-centering arrangement, stresses on the patient interface and laser unit are reduced when the handles are moved to place the grip in its “closed” configuration. A pair of brackets is used for this purpose. Structurally, each bracket is a curved member which is fixedly mounted on a support that is affixed to the base member. As so supported, the brackets extend part way around the periphery (circumference) of the orifice. In their positions, the brackets are situated across the orifice from each other, and they are located just outside the periphery of the orifice. When the patient interface is inserted into the orifice of the base member, the brackets pre-center the patient interface in the orifice. Subsequently, when the handles of the device are moved to establish the device in its closed configuration, the handles urge the respective brackets toward each other and against the patient interface.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic presentation of the components of a laser system for performing ophthalmic laser surgery on an eye of a patient, with portions shown in cross section or cut away for clarity;

FIG. 2 is a perspective view of an interface device in accordance with the present invention;

FIG. 3 is an exploded, perspective view of an interface device of the present invention;

FIG. 4 is a plan view of the latching and release mechanisms of the present invention; and

FIG. 5 is a perspective view of an alternate embodiment of the present invention.

### DESCRIPTION OF THE PREFERRED EMBODIMENTS

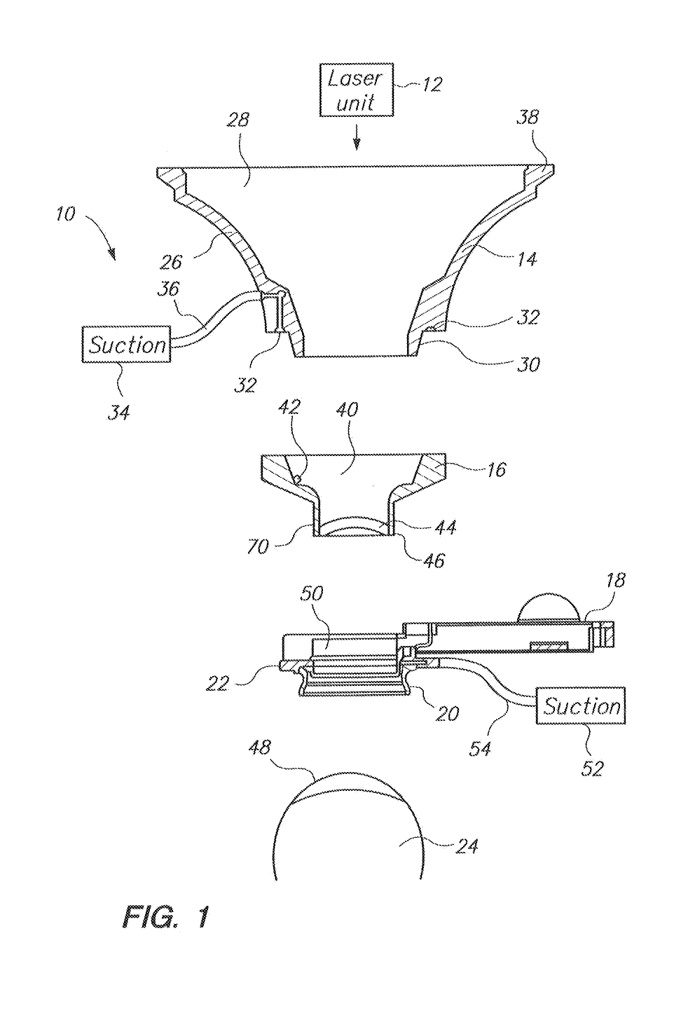

Referring initially to FIG. 1, the components of a system in accordance with the present invention are shown and collectively designated 10. As shown, the system 10 includes a laser unit 12 and an alignment device 14 that can be fixedly mounted on the laser unit 12. Further, the system 10 includes a patient interface 16 and a grip 18. As envisioned for the present invention, a suction ring 20 is attached to a base member 22, for example, by an interference fit attachment, and the base member 22, in turn, is operably coupled to the grip 18, as further detailed below. Operationally, during a surgical procedure, the suction ring 20 is positioned on the eye 24 of a patient, and the interface 16 is engaged between the alignment device 14 and the base member 22. One object of this structural combination is to position and maintain the eye 24 of a patient at a predetermined distance from the laser unit 12 during the surgical procedure.

FIG. 1 further shows that the alignment device 14 includes a wall 26 that surrounds an open passageway 28. Further, the wall 26 is formed with a tapered insert 30, and it has a suction channel 32 that is connected in fluid communication with a suction device 34 via a hose 36. Preferably, the taper of the insert 30 will be in a range between 14° and 22°. As stated above, and indicated in FIG. 1, the end 38 of alignment device 14 can be fixedly mounted on the laser unit 12. This can be accomplished in any manner well known in the pertinent art.

Still referring to FIG. 1 it will be seen that the patient interface 16 is formed with an open passageway 40 and includes a tapered receptacle 42. For the system 10 of the present invention, the taper of receptacle 42 in the patient interface 16 is compatible with the taper of the insert 30 on the alignment, device 14. This compatibility allows for a mating engagement of the alignment device 14 with the patient interface 16.

FIG. 1 also shows that a contact lens 44 is mounted at the end 46 of the patient interface 16 and, thus, is presented as an integral part of the patient interface 16. With this in mind, it is to be appreciated that the contact lens 44 and the patient interface 16 can be manufactured as a one-piece unit. The consequence of all this is that when the alignment device 14 is joined with the patient interface 16, the end 46 of the patient interface 16 (i.e. lens 44) will be at a predetermined distance from the end 38 of alignment device 14 (i.e. laser unit 12). Further, the contact lens 44 can be flat or curved, and for some applications, can be manufactured with a radius of curvature that generally conforms with the anterior surface 48 of the eye 24. FIG. 1 also shows that the suction ring 20 is connected in fluid communication with a suction, device 52, via a hose 54.

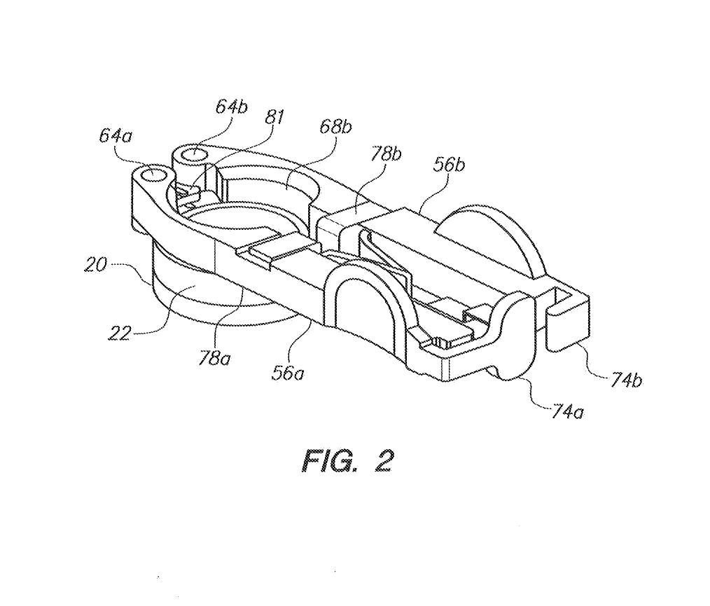

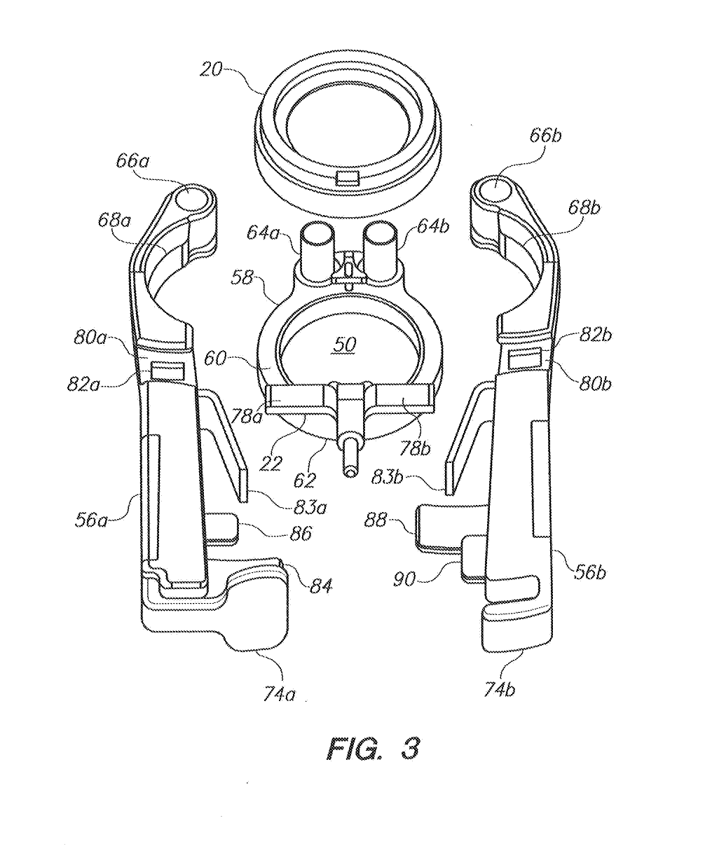

FIGS. 2 and 3 show the device of the present invention. As shown, the device includes base member 22, suction ring 20 and a pair of handles 56a,b (which together constitute the grip 18 shown and labeled in FIG. 1). For the present invention, the base member 22 has an annular shaped portion 58 that surrounds an orifice 50 that extends through the base member 22. It can further be seen that the base member 22 has a first side 60 and a second side 62. Geometrically, the orifice 50 is dimensioned, at the second side 62 to receive and hold the suction ring 20 against the base member 22 with an interference fit attachment. For the patient interface device 16, the suction ring 20 can be made of an elastomeric material and the base member 22 and handles 56a,b are typically made of a rigid medical grade plastic.

Continuing with FIGS. 2 and 3, it can be seen that two spaced-apart pivot posts 64a,b are mounted on the first side 60 of the base member 22 and extend parallel to each other in a direction perpendicular from a plane defined by the annular portion 58 of the base member 22. Also, each handle 56a,b is formed with a cylindrical through-hole 66a,b sized to allow each handle 56a,b to fit snuggly on a respective post 64a,b as shown in FIG. 2, and rotate about the post 64a,b and relative to the base member 22.

FIGS. 2 and 3 also show that each handle 56a,b is formed with an arcuate gripping surface 68a,b that is formed on each handle 56a,b such that when the handles 56a,b are rotated toward each other, the gripping surfaces 68a,b will independently; but cooperatively, contact the cylindrical surface 70 of the patient interface 16 which is rigidly attached to the laser unit 12 (see FIG. 1). With this arrangement, neither of the handles 56a,b will apply an effective force on the patient interface 16 (or laser unit 12) until they are both in contact with the patient interface 16. Once both handles 56a,b are in contact with the patient interface 16, however, the handles 56a,b will symmetrically apply equal and opposite forces against the patient interface 16 (and laser unit 12). As intended for the present invention, this cooperation of structure is provided to avoid the application of uneven or unequal forces against the laser unit 12 during an engagement of the suction ring 20 with the laser unit 12.

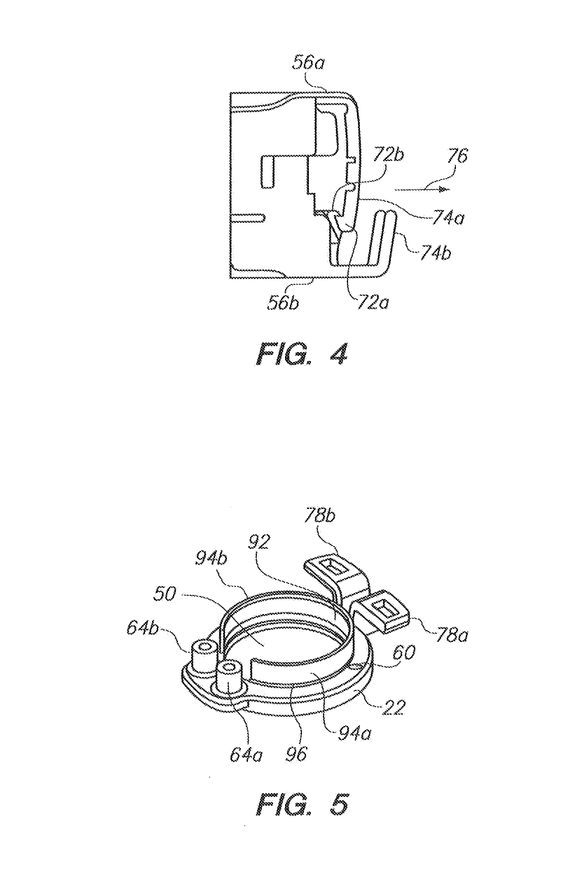

FIG. 4 shows that a latching mechanism can be included that has a hook-shaped latch member 72a formed near the end of a cantilever 74a that is formed on handle 56a and a complementary hook-shaped latch member 72b formed on handle 56b. To latch the handles 56a,b, the handles 56a,b are closed causing the end of the cantilever 74a to elastically displace slightly in the direction of arrow 76 until the latch member 72a passes the latch member 72b at which point the end of cantilever 74a relaxes and presses against handle 56b in a direction opposite the direction indicated by arrow 76. To unlatch the handles 56a,b, cantilever 74a can be slightly displaced in the direction of arrow 76 before opening the handles 56a,b. Cantilever 74b formed on handle 56b allows the user to unlatch the handles 56a,b by pressing the two cantilevers 74a,b together.

Cross-referencing FIGS. 1, 2 and 4, it can be seen the latch members 72a and 72b engage each other to hold the patient interface device 16 in a “closed” configuration in which the respective arcuate gripping surfaces 68a,b of the handles 56a,b are in contact with the cylindrical surface 70 of the patient interface 16 to firmly hold the patient interface device 16 on the laser unit 12. When the latch members 72a and 72b are disengaged, the handles 56a,b can be separated to place the device into an “open” configuration. In this “open” configuration the respective arcuate gripping surfaces 68a,b are distanced from the cylindrical surface 70 of the patient interface 16 to release the patient interface device 16 from the laser unit 12.

Returning to FIG. 3, it can be seen that a pair of wing arms 78a,b are mounted on the first side 60 of the base member 22. It can further be seen that the wing arms 78a,b are located diametrically across the orifice 50 from the pivot posts 64a,b, and they respectively extend away from each other in opposite directions tangential to the orifice 50. During assembly, each handle 56a,b receives one of the wing arms 78a,b in a respective slot 80a,b that is located on a respective handle 56a,b adjacent to its arcuate gripping surface 68a,b. For this combination, each wing arm 78a,b is formed with a protrusion on its underside (protrusion and underside not shown) for engagement with a hole 82a,b formed in a respective slot 80a,b. FIG. 2 shows an assembled device with the wing arms 78a,b positioned in the slots 80a,b (see FIG. 3). The interaction of a protrusion on a wing arm 78a,b, with a hole 82a,b (also see FIG. 3) in the slot 80a,b functions to limit the extent to which the handles 56a,b can separate from each other when the device is in its “open” configuration. Further, handles 56a,b are held in place by the combination of their position under tab 81 and the wing arms 78a,b in slots 80a,b which together restrict movement such that the handles 56a,b remain under the tab 81.

As best seen in FIG. 3, the handles 56a,b can each be formed with an extension spring 83a,b that interact together to bias the handles 56a,b apart and toward an “open” configuration for the patient interface device 16. Specifically, the extension springs 83a,b contact with each other when the patient interface device 16 is closed and generate a spring force that acts in opposition to the latching mechanism described above.

FIG. 3 also shows that the device can include two flange units. Specifically, handle 56a is formed with a first flange unit having a long flange 84 and a short flange 86 and handle 56b is formed with a second flange unit having a long flange 88 and a short flange 90. Thus, long, flange 84 interacts with short flange 90 and long flange 88 interacts with short flange 86. Also shown, on each handle 56a,b, the flanges are mounted adjacent and substantially parallel to each other. Further, the flanges are arranged so that when the handles 56a,b are closed, the long flange of one handle overlaps and contacts the short flange of the other and vice-versa. With the mutually overlapping engagement of the flange units, torsional movements between the handles 56a,b are prevented. The flange units can be provided to prevent, or minimize, any torsional forces that might otherwise be generated by the patient interface device 16 against the laser unit 12.

Referring now to FIG. 5, another embodiment is shown for pre-centering the patient interface 16 (see FIG. 1) with the orifice 50 of the base member 22 before the handles 56a and 56b (see. FIG. 2) are moved to place grip 18 in its “closed” configuration. With this pre-centering arrangement, stresses on the patient interface 16 and laser unit 12 are reduced when the handles 56a and 56b (see FIG. 2) are moved to place grip 18 in its “closed” configuration. In greater structural detail, as shown in FIG. 5, the pre-centering embodiment includes a vertical support 92 that is affixed to the first side 60 of the base member 22. As further shown, a pair of curved brackets 94a and 94b is affixed to the support 92, and the brackets 94a and 94b are situated across the orifice 50 from each other. As shown, the brackets 94a and 94b extend from the support 92 and over space 96 between the respective brackets 94a,b and base member 22, and they are respectively located adjacent the periphery of the orifice 50. When the patient interface 16 (see FIG. 1) is inserted into the orifice 50 of the base member 22, the brackets 94a,b pre-center the patient interface 16 in the orifice 50. Next, the handles 56a and 56b are moved to place grip 18 in its “closed” configuration. When this is done, the handles 56a and 56b urge the respective brackets 94a and 94b toward each other and into contact with surface 70 of the patient interface 16.

While the particular Improved Corneal Contact System as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. An interface device for selectively engaging a suction ring with a patient interface of a laser unit which comprises:
an annular shaped base member formed with an orifice, wherein the base member defines a plane and has a first side and a second side with the suction ring affixed against the second side of the base member to surround the orifice; and
a grip mounted on the first side of the base member, wherein the grip includes independently manipulated handles for symmetrically applying equal and opposite forces against the patient interface during an engagement of the device with the patient interface, to hold the base member and the suction ring, on the patient interface of the laser unit.

2. An interface device as recited in claim 1 wherein the grip comprises:
a pair of parallel pivot posts mounted side-by-side on the first side of the base member to extend therefrom perpendicular to the plane of the base member;
a first handle having a first end and a second end, wherein the first end is engaged with one pivot post for rotation of the first handle on the base member, and wherein the first handle is formed with an arcuate grip adjacent the first end; and
a second handle having a first end and a second end, wherein the first end is engaged with the other pivot post for rotation of the second handle on the base member, wherein the second handle is formed with an arcuate grip adjacent the first end, and wherein the first and second handles are rotated toward each other for respective contact with the patient interface of the laser unit to hold the base member and the suction ring in an engagement with the patient interface of the laser unit.

3. An interface device as recited in claim 2 further comprising:
a latch means including a first latch member mounted on the first handle for selective engagement with a second latch member-mounted on the second handle, to hold the interface device in a first configuration wherein the respective arcuate grips of the first and second handles contact the patient interface of the laser unit to firmly hold the interface device on the patient interface of the laser unit; and
a release means including a cantilever extending, from the second end of the first handle toward the second end of the second handle and a tab formed at the end of the cantilever, wherein the first latch member is mounted on the tab and the cantilever is biased to engage the first latch member with the second latch member, and further wherein the tab can be pulled to disengage the first latch member from the second latch member to move the device into a second configuration wherein the respective arcuate grips of the first and second handles are distanced, from the patient interface of the laser unit to release the interface device from the patient interface of the laser unit.

4. An interface device as recited in claim 3 further comprising:
a pair of wing arms mounted on the first side of the base member and located diametrically across the orifice from the pivot posts, wherein the wing arms respectively extend from the base member in opposite tangential directions from each other, wherein each wing arm is formed with a protrusion and each handle is formed with a slot having a hole for receiving a respective protrusion therein for interaction therewith to limit separation of the handles when the device is in the second configuration;
a first extension spring mounted on the first handle between its slot and its second end; and
a second extension spring mounted on the second handle between its slot and its second end, wherein the first extension spring and the second extension spring are each oriented for contact with each other to generate a spring force in opposition to the latch means, when the device is in the first configuration, to provide for a movement of the device into the second configuration in response to an activation of the release means.

5. An interface device as recited in claim 3 further comprising:
a pair of wing arms mounted on the first side of the base member and located diametrically across the orifice from the pivot posts, wherein the wing arms respectively extend from the base member in opposite tangential directions from each other, wherein each wing arm is formed with a protrusion and each handle is formed with a hole for receiving a respective protrusion therein for interaction therewith to limit separation of the handles when the device is in the second configuration;
a first arcuate bracket mounted on the base member, wherein the first bracket is situated adjacent a peripheral portion of the orifice; and
a second arcuate bracket mounted on the base member opposite the orifice from the first bracket, wherein the second bracket is situated adjacent a peripheral portion of the orifice, wherein the first bracket and the second bracket are each situated for contact with the patient interface to pre-center the patient interface relative to the orifice before the handles engage the patient interface.

6. An interface device as recited in claim 4 further comprising:
a first flange unit affixed to the first handle between its slot and the second end thereof, to extend therefrom toward the second handle; and
a second flange unit affixed, to the second handle between its slot and the second end thereof, to extend therefrom toward the first handle and establish a mutually overlapping engagement with the first flange unit, for prevention of a torsional movement between the first handle and the second handle, wherein each flange unit has a long flange and a short flange, wherein each short flange is mounted adjacent and substantially parallel to its respective long flange, and wherein the overlapping engagement of the first flange unit with the second flange unit causes the long flange of each respective flange unit to overlap the short flange of the other flange unit.

7. An interface device for selectively engaging a suction ring with a patient interface of a laser unit which comprises:
an annular shaped base member formed with an orifice, wherein the base member defines a plane and has a first side and a second side, the suction ring affixed against the second side of the base member to surround the orifice;
a pair of parallel pivot posts mounted side-by-side on the first side of the base member to extend therefrom perpendicular to the plane of the base member;
a first handle having a first end and a second end, wherein the first handle is formed with an arcuate grip adjacent the first end, and wherein the first end is engaged with a pivot post for rotation of the first handle on the base member; and
a second handle having a first end and a second end, wherein the second handle is formed with an arcuate grip adjacent the first end, wherein the first end is engaged with the other pivot post for rotation of the second handle on the base member, and wherein the first and second handles are rotated toward each other for respective contacts with the patient interface to hold the base member and the suction ring in an engagement with the patient interface of the laser unit.

8. A device as recited in claim 7 further comprising:
a latch means for holding the interface device in a first configuration wherein the respective arcuate grips of the first and second handles engage the patient interface to firmly hold the interface device on the patient interface of the laser unit; and
a release means selectively activated to disengage the latch means for movement of the interface device from the first configuration and into a second configuration, wherein the respective arcuate grips of the first and second handles are distanced from the patient interface to release the interface device from the patient interface of the laser unit.

9. A device as recited in claim 8 wherein the latch means comprises:
a first latch member mounted on the first handle; and
a second latch member mounted on the second handle for selective engagement of the second latch with the first latch to hold the interface device in the first configuration.

10. A device as recited in claim 9 wherein the release mean comprises:
a cantilever extending from the second end of the first handle toward the second end of the second handle; and
a tab formed at the end of the cantilever, wherein the first latch member is mounted on the tab and the cantilever is biased to engage the first latch member with the second latch member, and wherein the tab can be pulled to disengage the first latch member from the second latch member to move the device into its second configuration.

11. A device as recited in claim 8 further comprising a pair of wing arms mounted on the first side of the base member and located diametrically across the orifice from the pivot posts, wherein the wing arms respectively extend from the base member in opposite tangential directions from each other, wherein each wing arm is formed with a protrusion and each handle is formed with a slot having a hole for receiving a respective protrusion therein for interaction therewith to limit separation of the handles when the device is in the second configuration.

12. A device as recited in claim 11 further comprising:
a first extension spring mounted on the first handle between the slot and the second end thereof; and
a second extension spring mounted on the second handle between the slot and the second end thereof, wherein the first extension spring and the second extension spring are each oriented for contact with the other extension spring to generate a spring force in opposition to the latch means when the device is in the first configuration, to provide for a movement of the device into the second configuration in response to an activation of the release means.

13. A device as recited in claim 11 further comprising:
a first flange unit affixed to the first handle between its slot and the second end thereof, to extend therefrom toward the second handle; and
a second flange unit affixed to the second handle between its slot and the second end thereof, to extend therefrom toward the first handle and establish a mutually overlapping engagement with the first flange unit, for prevention of a torsional movement between the first handle and the second handle.

14. A device as recited in claim 1 wherein each flange unit comprises:
a long flange; and
a short flange, wherein the short flange is mounted adjacent and substantially parallel to the long flange, and wherein the overlapping engagement of the first flange unit with the second flange unit causes the long flange of each respective flange unit to overlap the short flange of the other flange unit.

15. A device as recited in claim 7 wherein the suction ring is made of an elastomeric material.

16. A device as recited in claim 7 wherein the base member comprises a suction port to establish fluid communication with a suction channel of the suction ring when the suction ring is held against the base member.

17. A device as recited in claim 7 wherein the device is made of a medical grade plastic.

18. A method for manufacturing an interface device for selectively engaging a suction ring with a patient interface of a laser unit which comprises the steps of:
providing an annular shaped base member formed with an orifice, wherein the base member defines a plane and has a first side and a second side, with the suction ring affixed against the second side of the base member to surround the orifice;
mounting a pair of parallel pivot posts side-by-side on the first side of the base, member to extend therefrom perpendicular to the plane of the base member;
engaging a first handle with a pivot post for rotation of the first handle on the base member, wherein the first handle has a first end and a second end and is formed with an arcuate grip adjacent the first end;
engaging a second handle with the other pivot post for rotation of the second handle on the base member, wherein the second handle has a first end and a second end and is formed with an arcuate grip adjacent the first end, and wherein the first and second handles can be rotated toward each other for respective engagement with the patient interface to hold the base member and the suction ring in an engagement with the patient interface of the laser unit;
creating a latch means on the device for holding the interface device in a first configuration wherein the respective arcuate grips of the first and second handles engage the patient interface to firmly hold the interface device on the patient interface of the laser unit; and
forming a release means on the device for selectively disengaging the latch means for movement of the interface device from the first configuration and into a second configuration, wherein the respective arcuate grips of the first and second handles are distanced from the patient interface to release the interface device from the patient interface of the laser unit.

19. A method as recited in claim 18 further comprising the steps of:
positioning a first extension spring on the first handle; and
positioning a second extension spring on the second handle, wherein the first extension spring and the second extension spring are each oriented for contact with each other to generate a spring force in opposition to the latch means, when the device is in the first configuration, and to provide for a movement of the device into the second configuration in response to an activation of the release means.

20. A method as recited in claim 18 wherein the suction ring is made of an elastomeric material and the device is made of a medical grade plastic.
